(19) United States
(12) Patent Application Publication
(10) Pub. No.: US 2009/0306563 A1
(43) Pub. Date: Dec. 10, 2009

(54) CERVICAL SPINE TRACTION COLLAR

(76) Inventor: David J. BEN-KIKI, Nahariya (IL)

Correspondence Address: BLANK ROME LLP, WATERGATE, 600 NEW HAMPSHIRE AVENUE, N.W., WASHINGTON, DC, 20037, US

(21) Appl. No.: 12/482,046

(22) Filed: Jun. 10, 2009

(30) Foreign Application Priority Data
Jun. 10, 2008 (IL) 192038

(51) Int. Cl. A61F 5/00

(52) U.S. Cl. 602/18

(57) ABSTRACT

A cervical spine traction collar is disclosed. The cervical spine collar comprises at least two cervical spine embracing elements, which are relatively displaceable with respect to each other in either up or down direction; a displacement device for relatively displacement of the cervical spine embracing elements in the up or down direction; a fastening device for securing the cervical spine embracing elements in a position in which they embrace a user's neck; and a padding provided on said cervical spine embracing elements for soft contact between the spine embracing elements and a patient body.

### FIELD OF THE INVENTION

The present invention relates to a new and useful cervical spine traction collar (CSTC). More particularly, the present invention relates to a CSTC for providing at least one of stabilization to an injured area in the cervical spine, reduction of acute or chronic neuralgia, and treatment for Acute Traumatic conditions at an accident site.

### BACKGROUND OF THE INVENTION

In the health care field today, cervical collars in general are used to limit Range of Motion of the cervical spine following traumatic conditions such as Spastic Torticollis, dislocations, post traumatic vertebral fractures (e.g., Lamina, Pedicals, collapsed body, etc.), degenerative changes interfering with neurological activity and conduction, resulting from disc degeneration, causing a reduction in the intervertebral space. The majority of cervical collars offer limited help to the above-mentioned conditions since they mainly reduce range of motion to the cervical musculosketal complex without capability to make any correction or to address pure traction within the cervical spine.

Some traditional collars are made with inflatable bladders that, when inflated, are designed to lift the head off of the patient's shoulders. The weight of the head could possibly be heavier than the load that the air bladders could handle. Moreover, in the event that only one side of the neck is injured and requires more tractions than the other side of the neck, such a cervical collar cannot provide unilateral correction. Accordingly, there is a need for a cervical spine traction collar for treating the above-mentioned conditions while providing the capability to address pure traction within the cervical spine and to make unilateral corrections.

### SUMMARY OF THE INVENTION

Accordingly, to solve at least the above problems and/or disadvantages and to provide at least the advantages described below, a non-limiting object of the present invention is to provide a cervical spine collar that comprises at least two cervical spine embracing elements, which are relatively displaceable with respect to each other in either up or down direction; a displacement means for relatively displacement of the cervical spine embracing elements in the up or down direction; a fastening means for securing the cervical spine embracing elements in a position in which they embrace a user's neck; a padding means can be provided on said cervical spine embracing elements for soft contact between the spine embracing elements and a patient body. These and other objects of the invention, as well as many of the intended advantages thereof, will become more readily apparent when reference is made to the following description, taken in conjunction with the accompanying drawings.

### BRIEF DESCRIPTION OF THE DRAWINGS

FIG. 1 is an orthogonal view illustrating a non-limiting exemplary embodiment of a Cervical Spine Traction Collar (CSTC) according to the present invention;

FIG. 2A is a front elevational view illustrating the CSTC of FIG. 1 with its internal element in a retracted position;

FIG. 2B is a front elevational view illustrating the CSTC of FIG. 1 with its internal element in a protracted position;

FIG. 3A is a rear elevational view illustrating the CSTC of FIG. 1 with its internal element in a retracted position and with its fastening straps fastened;

FIG. 3B is a rear elevational view illustrating the CSTC of FIG. 1 with its internal element in a protracted position and with its fastening straps fastened;

FIG. 4 is an elevational illustrating the CSTC of FIG. 1 in a completely open and fully protracted position;

FIG. 5 is a partially exploded view illustrating the upper portion of the CSTC of FIG. 4;

FIG. 6 is a partially exploded view illustrating the lower portion of the CSTC of FIG. 4;

FIG. 7 is front elevational view with an enlarged view illustrating the retaining knob and rack of the CSTC of FIG. 1;

FIG. 8 is a front and side elevational view illustrating another non-limiting exemplary embodiment of a Cervical Spine Traction Collar (CSTC) according to the present invention;

FIG. 9 is a front elevational view illustrating the CSTC of FIG. 1 secured around a patient's neck with its internal element in a retracted position;

FIG. 10 is a front elevational view illustrating the CSTC of FIG. 1 secured around a patient's neck with its internal element in a protracted position;

FIG. 11 is a rear elevational view illustrating the CSTC of FIG. 1 secured around a patient's neck with its internal element in a protracted position and with its fastening straps fastened; and

FIG. 12 is a front elevational view illustrating the CSTC of FIG. 1 secured around a patient's neck with one side of its internal element in a protracted position to provide unilateral correction.

### DETAILED DESCRIPTION OF THE INVENTION

Reference will now be made in detail to non-limiting embodiments of the present invention by way of reference to the accompanying drawings, wherein like reference numerals refer to like parts, components, and structures.

The Cervical Spine Traction Collar (CSTC) 1 of the present invention is a new design that addresses a wide range of neck problems/injuries. It incorporates functionality developed based on a detailed biomechanical understanding of the cervical spine musculoskeletal complex. Unlike traditional cervical collars, the CSTC 1 of the present invention provides a telescopic action built into the collar that provides the desired traction necessary regardless of the patient's physical location (e.g., the field, work, car, etc.). In the event of an acute trauma to the cervical musculoskeletal complex, use of the CSTC 1 of the present invention helps to reduce and restore the subdural space, thus allowing a continuous flow of cerebral spinal fluid with the subdural space. This reduces the odds of edema build-up in the menengies and along the spinal cord.

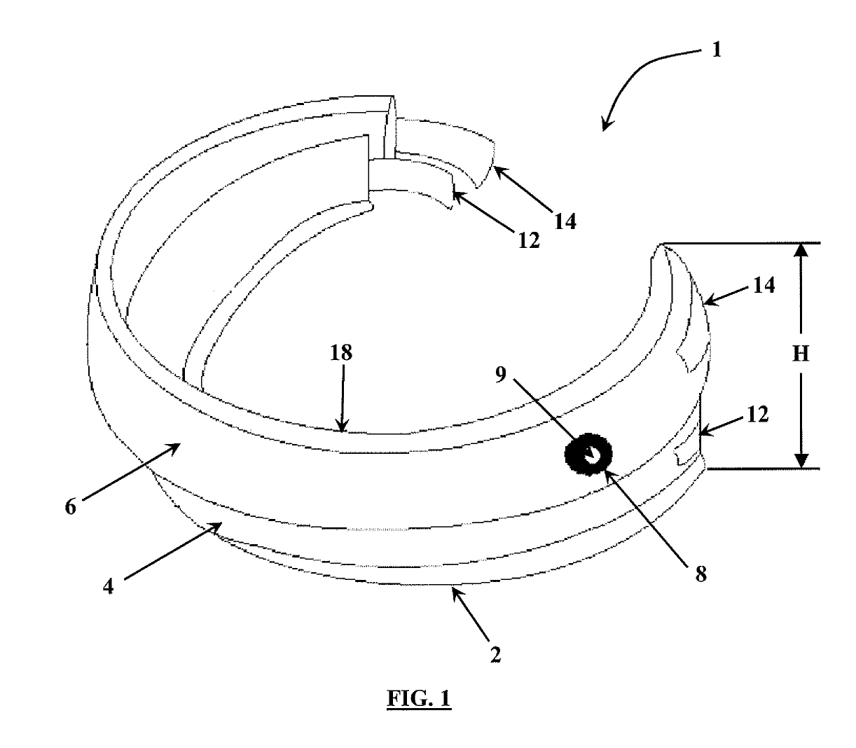

Turning to the figures, FIG. 1 illustrates a CSTC 1 according to a non-limiting embodiment of the present invention. The CSTC includes an internal element 4, an external element 6, lower padding 2, upper padding 18, a plurality of adjustment knobs 8, and a plurality of fastening straps 12 and 14. The internal element 4 is configured to be slidably disposed within the external element 6 in a telescoping manner. The lower padding 2 is disposed on a lower portion of the internal element 4 and the upper padding 18 is disposed on an upper portion of the external element 6 to provide support and comfort for a user. The adjustment knobs 8 provide functionality for adjusting the position of the internal element 4 within the external element 6 to adjust the overall height “H” of the CSTC 1, which will apply varying degrees of cervical traction to a user's spine based on that height “H”. At least one pair of fastening straps 12 is disposed on opposing ends of the internal element 4 at about half the distance between the top and bottom edges. And, at least one pair of fastening straps 14 is disposed on opposing ends of the external element 6 to fasten the CSTC 1 at about one third the distance from the bottom edge to the top edge of the internal element 4. Fastening the fastening straps 12 and 14 secures the internal and the external element of the CSTC 1 in a position, in which they embrace a user's neck.

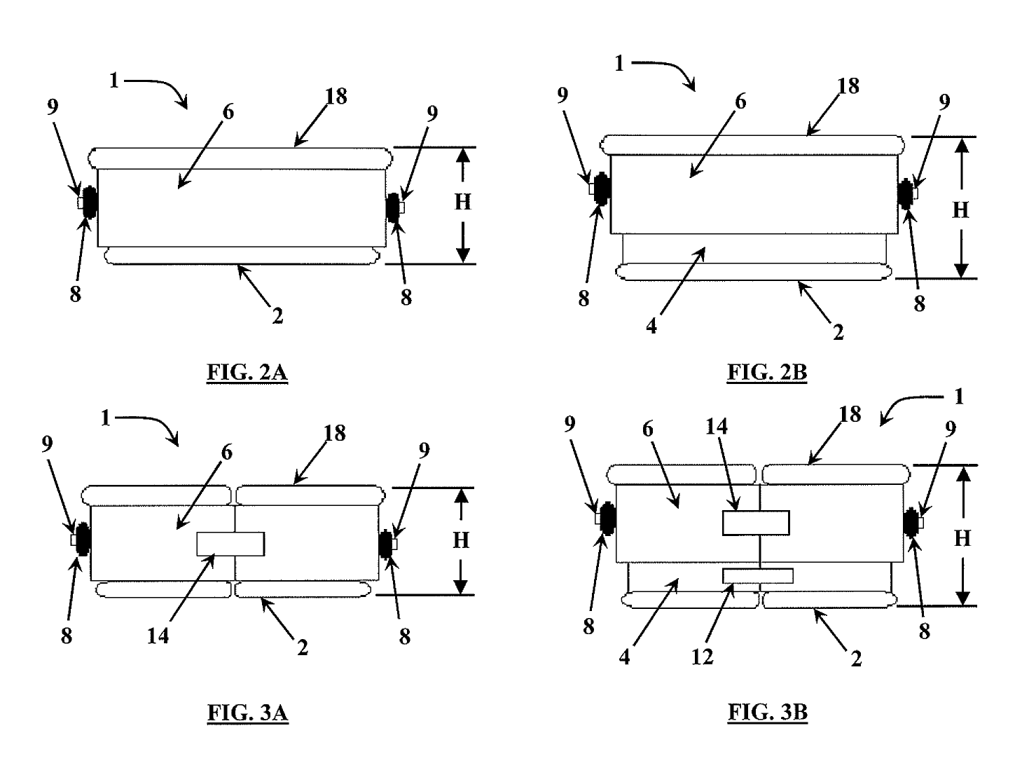

FIG. 1 illustrates the CSTC 1 in the “open” position. When in the open position, both the internal element 4 and the external element 6 substantially take the form of a “C” shape such that there is provided an opening in which a user's neck can be inserted. FIGS. 2A-3B illustrate the CSTC 1 in the “closed” position—the position the CSTC 1 would be in when the straps are fastened to secure the CSTC around a user's neck. Both the internal element 4 and the external element 6 can be made of a durable, yet flexible, material so they may be wrapped around a user's neck and conform to the neck's cross-sectional shape when the CSTC 1 is placed around the user's neck. As FIGS. 2B and 3B illustrate, hook and loop portions (e.g., VELCRO brand hook and loop fasteners) of the fastening straps 12 and 14 when they are brought together to secure the CSTC 1 around the user's neck. When the hook and loop portions of the fastening straps 12 and 14 are brought together, the internal element 4 and the external element 6 substantially take the form of an “O” shape such that the user's neck is enclosed within the CSTC 1.

In the closed position, the adjustment knobs 8 are disposed on diametrically opposed sides of the CSTC 1 from each other. By turning the adjustment knobs 8, a user can adjust the height “H” of each respective side of the CSTC 1. FIGS. 2A and 3A illustrate the CSTC 1 with both sides of the internal element 4 retracted fully within the external element 6 to produce a minimum value of “H” (i.e., minimal traction). In the fully retracted position, only the lower padding 2 on the internal element 4 is visible. The internal element 4 and external element are 6 are configured so that the fastening straps 12 on the internal element 4 will not interfere with the slidable engagement of the internal element 4 and the external element 6.

FIGS. 2B and 3B illustrate the CSTC 1 with both sides of the internal element 4 protracted fully from the external element 6 to produce a maximum value of “H” (i.e., maximum traction). The dimensions of the internal element 4 and the external element 6 can be altered as required to provide different maximum heights “H” and, therefore, different maximum values of traction. Those dimensions can also be adjusted to suit the various neck sizes of different users.

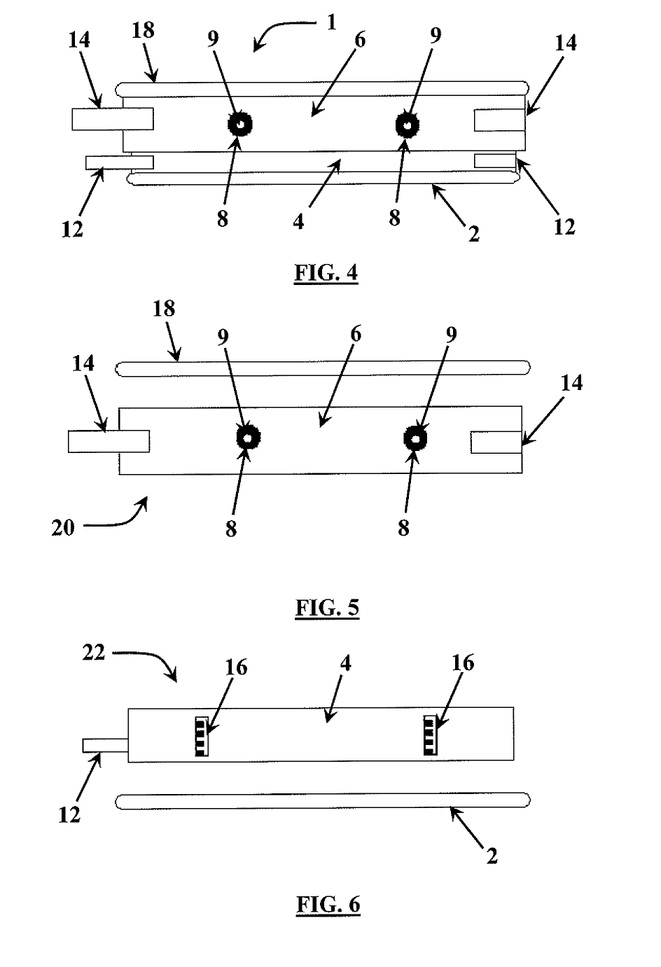

FIGS. 4-6 illustrate the CSTC 1 in a completely open and fully protracted position such that an entire side of the CSTC 1 is visible. In that position, the CSTC 1 is substantially straight in lieu of being in the shape of a “C” or an “O” as described above. FIG. 4 illustrates a fully assembled CSTC 1. FIG. 5 illustrates the upper half 20 of the CSTC 1, which includes the external element 6, the upper padding 18, and the adjustment knobs 8. FIG. 6 illustrates the lower half 22 of the CSTC 1, which includes the internal element 4, the lower padding 2, and the racks 16.

As FIGS. 5 and 6 illustrate, the upper padding 18 is of sufficient length to cover the entire upper edge of the external element 6 and the lower padding 2 is of sufficient length to cover the entire lower edge of the internal element 4. FIG. 5 illustrates the outward facing portion of the external element 6 with the adjustment knobs 8 extending outward therefrom, and FIG. 6 illustrates the inward facing portion of the internal element 4 with racks 16 disposed therein. Each of the racks 16 may be disposed in the internal element 4 at approximately one quarter of the length of the internal element 4 from each corresponding distal end of the internal element.

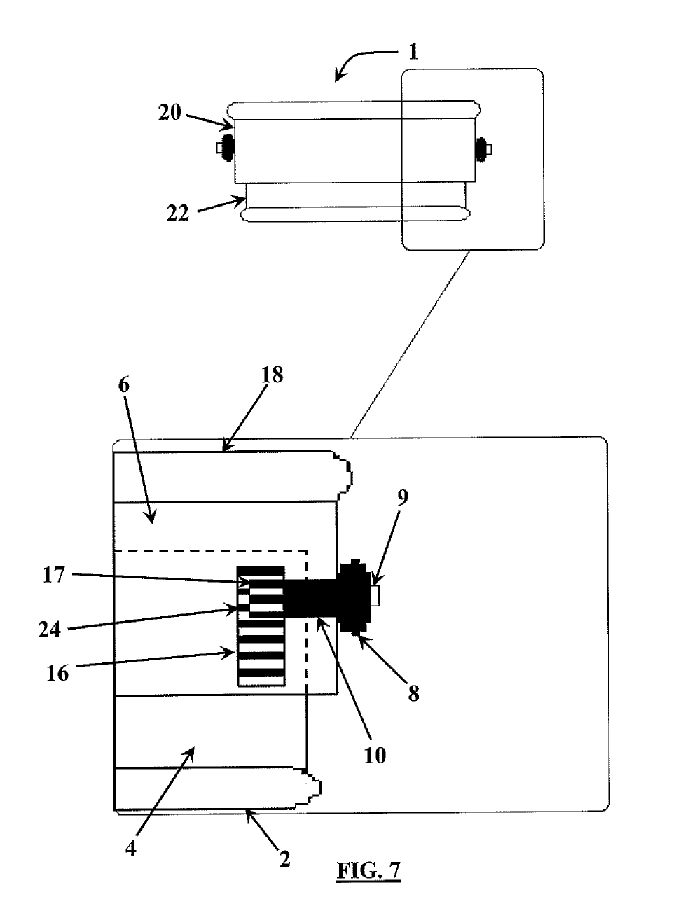

As FIG. 7 illustrates, the adjustment knobs 8 are disposed at a location on the external element 6 that corresponds with the location of the racks 16 of the internal element 4 so that the alignment knobs 8 can engage the racks 16 when the internal element 4 is slidably disposed in the external element 6. Each adjustment knob 8 includes a substantially cylindrical shaft 10 that extends through the external element 6. The distal end of the shaft 10 includes a plurality of teeth 17 that extend laterally outward from the shaft 10. Teeth 24 are provided on the rack 16 and teeth 17 of the shaft 10 are configured to mesh with corresponding teeth 24 of each rack 16 in a rack-and-pinion configuration. The shaft 10 is received in the rack 16 so that the teeth 17 of the shaft 10 engage the teeth 24 of the rack 16. When the teeth 17 on the shaft 10 of the adjustment knob 8 are meshed with the teeth 24 in each rack 16, the adjustment knobs 8 can be rotated to move the external element 6 up and down the racks 16 relative to the internal element 4.

Accordingly, the knobs 8 adjust the height of the collar 1 by slidable displacement the external element 6 with respect to the internal element 4. The internal and external elements 4, 6 are elongated and each have a longitudinal axis. The elements 4, 6 are configured such that when the internal element is in fully retracted position their longitudinal axes are substantially parallel to one another. The knobs 8 and rack 16 cooperate to move the elements 4, 6 in a direction orthogonal to the longitudinal axes, thereby adjusting the height of the collar 1. In addition, the collar 1 can be positioned at any desirable height, and is not limited to only predefined heights.

The adjustment knobs 8 may also include locking functionality so that the shaft 10 of the adjustment knob 8 will not turn after the internal element 4 is protracted or retracted to the desired position. The locking functionality can for example include a cog such that when the knob 8 is pulled, it disengages a cog, which allows the user to turn the knob to extend or retract the collar. A button 9 can be provided on each adjustment knob 8 such that when it is depressed the adjustment knob 8 is locked into position and this prevents the shaft 10 from being rotated. Other locking arrangements can be provided as well. For instance, the knob 8 can be pulled out to position the collar 1, and then pushed in to lock the collar 1 at one of the 7 incremental steps provided by the teeth 17, 24

Such locking functionality allows a user to maintain the position of the internal element 4 with respect to the external element 6 for prolonged periods without continued adjustment to maintain traction. Accordingly, the adjustment knob 8 provides functionality for a user to incrementally retract and protract the CSTC 1 and to lock in the amount of traction once the amount of protraction is properly adjusted. A larger number of teeth 17 and 24 can be provided on each adjustment knob 8 and rack 16, respectively, to allow more fine incremental adjustments with the adjustment knobs 8.

In a preferred embodiment of the CSTC 1, the external element 6 h as a length of approximately 51-54 cm and a width of approximately 8 cm; the internal element 4 has a length of approximately 49-52 cm and a width of approximately 5 cm; the lower padding 2 and upper padding 18 are each formed from soft/medium foam and have lengths substantially the same as the internal element 4 and external element 6, respectively, and a width of approximately 1.5-2 cm. Also in a preferred embodiment of the CSTC 1, the materials used to make the various elements of the CSTC 1 are technologically advanced and environmentally friendly. For example, natural rubber (NBR), polyurethane (EPU), or even silicone materials may be used for the internal and external elements 4, 6 and the lower and upper padding 2, 18. Those materials are light in weight and firm for necessary support and flexibility, which provides added comfort to the CSTC 1 and makes the CSTC 1 more user-friendly. Moreover, those materials may be “breathable,” which prevents the patient from over-heating while wearing the CSTC 1.

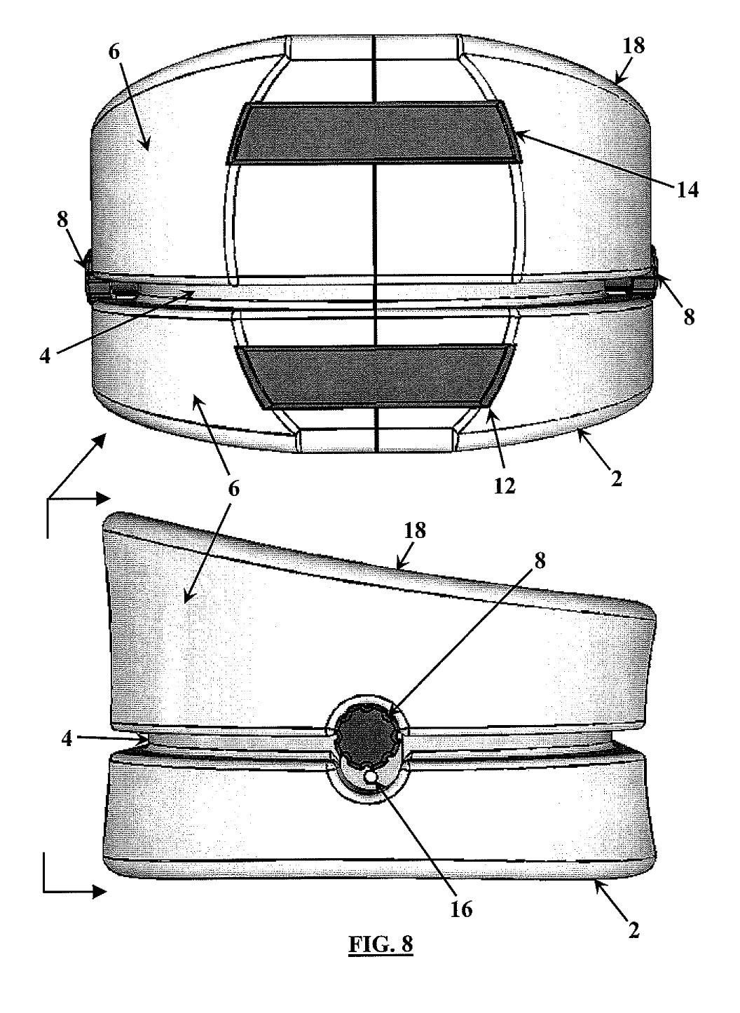

In FIGS. 1-7, the outer surface of the internal element 4 is shown as having a smaller diameter than the outer surface of the external element 6, such that the internal element 4 is recessed with respect to the external element 6. Referring to FIG. 8, however, the elements 4, 6 can have the same outer diameter by providing padding on the internal element 4 so that the outer surfaces of the internal element 4 and the external element 6 are flush with each other. As further shown in FIG. 8, the upper pad 18 can be tapered so that it is higher in the rear and lower in the front, to better conform to the user's head and neck, and make better traction with the user's spine.

In use, the CSTC 1 is placed around the user's neck and fastened by the two fastening straps 12 and 14 when the internal element 4 is fully retracted within the external element 6. One fastening strap 12 is attached to the internal element 4 and the other fastening strap 14 is attached to the external element 6. The adjustment knobs 8 of the external element 6 are gradually turned to protract the internal element 4 from behind the external element 6. That action creates the cervical spine traction, using the telescopic effect by virtue of the rack-and-pinion arrangement described with respect to FIG. 7. That arrangement provides is unique to the CSTC 1 of the present invention.

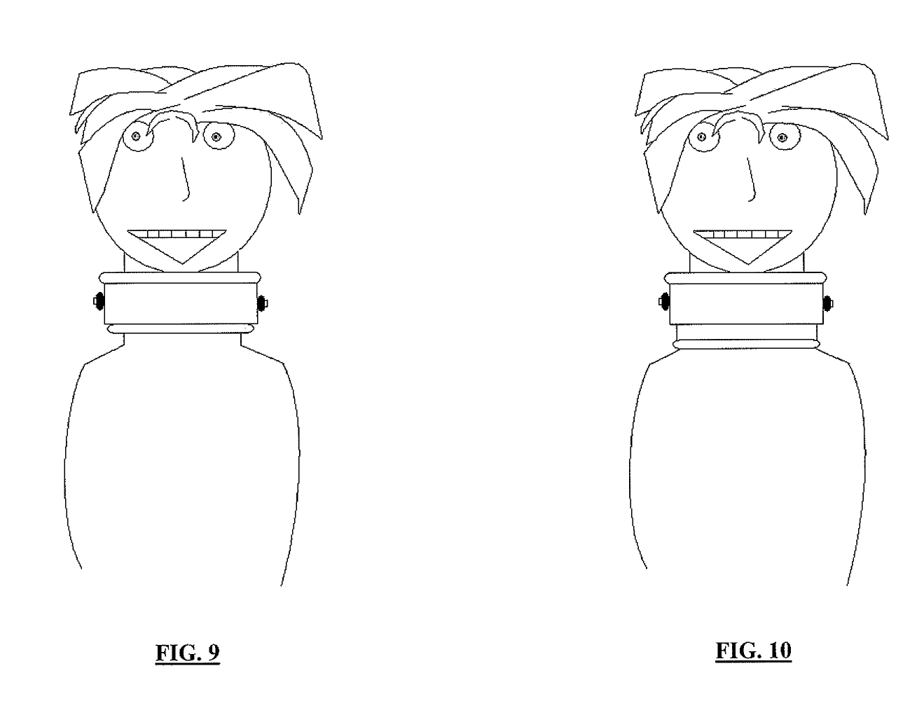

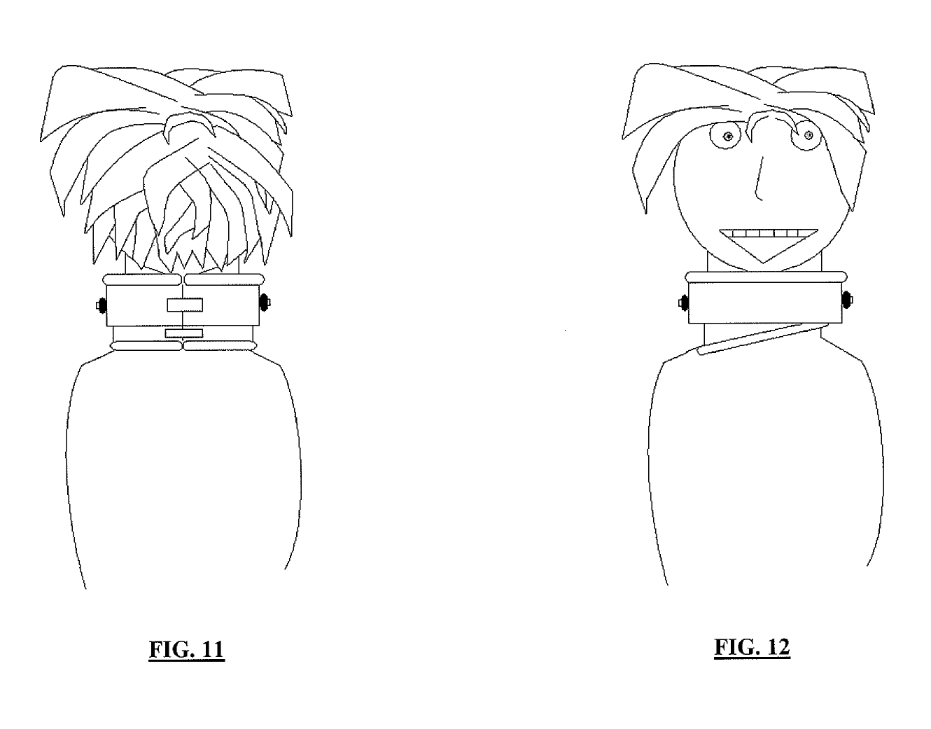

FIGS. 9-11 illustrate the CSTC 1 of the present invention being utilized to provide various forms of traction. FIG. 9 illustrates the internal element 4 in a retracted position so as to only apply little to no traction to a person in pain. FIG. 10 illustrates the internal element 4 in a protracted position to create a desired amount of traction effect to reduce nerve conduction interference. And, FIG. 11 illustrates the fastening straps 12 and 14 fastened on the rear of the CSTC 1 illustrated in FIG. 10.

Another unique feature of the CSTC 1 of the present invention is its ability to protract internal element 4 unilaterally (i.e., one side instead of both sides of the neck) for situations where one side of the neck may require more traction than the other side. FIG. 12 illustrates the internal element 4 protracted in a unilateral protracted position, allowing more traction to be applied to one side of the user's neck than the other if needed. That unilateral protraction functionality allows CSTC 1 to be used to address cervical spine musculoskeletal complex injuries that require such treatment.

The CSTC 1 of the present invention is a superior and unique cervical spine traction collar unlike any other in the health-care industry today. The materials used to make the CSTC 1 are light-weight and firm enough to reliably secure a user's neck while in traction. Those materials are also flexible and eco-friendly. The rack-and-pinion configuration allows the patient to gradually increase or decrease the degree of traction without causing undo stress to the cervical spine. The two C-shaped elements create a true traction to the cervical spine.

In addition, use of the CSTC 1 of the present invention is not limited to the health care industry. It can also be used in emergency situations by air or ground medi-vac services. Moreover, it is user friendly, which means that once the medical provider administers the CSTC 1 to his/her patient, the patient can use it easily at home or at work.

In the preferred embodiment, two sets of knobs 8 and respective racks 16 are provided on substantially opposite sides of the collar 1. Preferably, one set on the front and one set on the back, or the sets on the sides. Two sets of knobs 8 and respective racks 16 provide sufficient stability and adjustability to the collar 1. However, more or fewer than two sets of knobs 8 and racks 16 can be provided, within the spirit of the invention.

In the embodiments shown in FIGS. 1-18, the external element 6 is on top and the internal element 4 is on the bottom. It should be appreciated, however, that the collar can also be worn in the inverted position, namely with the internal element 4 on top and the external element 6 on the bottom.

The foregoing description and drawings should be considered as illustrative only of the principles of the invention The invention may be configured in a variety of shapes and sizes and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

What is claimed is:

1. An adjustable cervical spine collar comprising:
at least two cervical spine embracing elements, which are displaceable with respect to each other to adjust a height of said cervical spine collar;
a displacement device for displacing the cervical spine embracing elements with respect to each other to provide a desired height; and
a fastening device for securing the cervical spine embracing elements in a position in which they embrace a user's neck.

2. The cervical spine collar according to claim 1, wherein said at least two cervical spine embracing elements each have a longitudinal axis and are displaceable with respect to each other in a direction substantially perpendicular to the longitudinal axis.

3. The cervical spine collar according to claim 1, wherein said cervical spine embracing elements have a C-shaped configuration and said elements are elastically deformable.

4. The cervical spine collar according to claim 3, wherein said cervical spine embracing elements comprise an external C-shaped element and an internal C-shaped element, wherein upon relative displacement of the internal C-shaped element down with respect to the external C-shaped element, traction of the cervical spine can be affected.

5. The cervical spine collar according to claim 4, wherein said traction is initiated upon securing the internal element and the external element in a position in which they embrace a user's neck.

6. The cervical spine collar according to claim 1, wherein said displacement device comprises a rack-and-pinion arrangement.

7. The cervical spine collar according to claim 1, wherein said at least two cervical embracing elements are manufactured from a plastic material.

8. The cervical spine collar according to claim 7, wherein said plastic material is selected from a group consisting of a rubber and polyurethane.

9. The cervical spine collar according to claim 1, further comprising padding provided on said at least two cervical spine embracing elements.

10. The cervical spine collar according to claim 9, wherein said padding comprises a cushion provided at an edge of each of the cervical spine embracing elements.

11. The cervical spine collar according to claim 10, wherein said cushion is made of silicon based foamed material.

12. The cervical spine collar according to claim 1, wherein said fastening device comprises hook and loop fasteners provided on opposite ends of the cervical spine embracing elements.

13. The cervical spine collar according to claim 1, wherein said displacement device comprises a first displacement device positioned at one side of said cervical spine collar and a second displacement device positioned at an opposite side of said cervical spine collar, wherein said first displacement device and said second displacement device operate independently of one another to provide different traction the one side of said cervical spine collar than at the opposite side of said cervical spine collar.

14. An adjustable cervical spine collar comprising:
an external elongated collar portion defining an interior space;
an external engagement element connected to said external elongated collar portion;
an internal elongated collar portion, said external elongated collar portion and said internal elongated collar portion defining a height of said cervical spine collar; and
an internal engagement element associated with said internal elongated collar portion and configured to cooperate with said external engagement element, the internal engagement element and external engagement element cooperating to relatively displace said internal elongated collar portion with respect to said external elongated collar portion to adjust the height of said cervical spine collar.

15. The cervical spine collar according to claim 14, further comprising a fastening device for securing the elongated cervical spine embracing elements in a position in which they embrace a user's neck.

16. The cervical spine collar according to claim 14, wherein said external engagement element comprises a shaft having teeth and said internal engagement element comprises a rack having teeth, wherein the teeth of said shaft engage the teeth of said rack.

17. A method for causing traction of a cervical spine comprising:
providing at least two cervical spine embracing elements forming a cervical spine collar, the at least two cervical spine embracing elements displaceable with respect to each other to adjust a height of said cervical spine collar;
providing a displacement device for displacing the cervical spine embracing elements with respect to each other to provide a desired height; and
providing a fastening device for securing the cervical spine embracing elements in a position in which they embrace a user's neck.

18. The method according to claim 17, wherein said at least two cervical spine embracing elements each have a longitudinal axis and are displaceable with respect to each other in a direction substantially perpendicular to the longitudinal axis.

19. The method according to claim 17, further comprising providing padding on said cervical spine embracing elements.

20. The method according to claim 17, further comprising the steps of positioning the two embracing elements on the user's neck, fastening the two embracing elements to each other around the user's neck, displacing the two embracing elements relative to each other to adjust a height of the collar.
